(19) United States
(12) Patent Application Publication
(10) Pub. No.: US 2020/0380887 A1
(43) Pub. Date: Dec. 3, 2020

(54) Health Wellness Assessment and Management System

(71) Applicant: Nuberty, Inc., Sandy Springs, GA (US)

(72) Inventor: Desmond Mason, Sandy Springs, GA (US)

(21) Appl. No.: 16/426,155

(22) Filed: May 30, 2019

(51) Int. Cl. G09B 19/00; A61B 5/00; A61B 5/11; A61B 5/16; A61B 5/0205; G16H 10/20; G16H 20/30; G16H 20/10

(52) U.S. Cl. CPC G09B 19/0092; A61B 5/4869; A61B 5/1118; A61B 5/165; A61B 5/4872; A61B 5/021; A61B 5/0022; G16H 10/20; G16H 20/30; G16H 20/10; A61B 5/0205

(57) ABSTRACT

A health wellness assessment and management system for men that takes into account a “bio-wellness age” based in part on a subject's testosterone level. The system computes a subject's “current bio-wellness age” and compiles a customized plan for the subject that includes a testosterone replacement therapy and other target evaluation factors. The system also computes a “target bio-wellness age” assuming that the subject attains the target testosterone level by taking the testosterone replacement therapy, along with attaining the other target evaluation factors through lifestyle changes recommended in the customized plan. The subject's chronological age, current bio-wellness age, and target bio-wellness age are used as gamification parameters to motivate the subject to engage in a health wellness improvement program. For example, the system may show a chronological age of “56,” current bio-wellness age of “67,” and a target bio-wellness age of “32” as a motivational feature.

### TECHNICAL FIELD

The present invention is directed to health wellness systems and, more particularly, to a health wellness assessment and management system for men that computes a “bio-wellness age” based in part on a subject's testosterone level as a gamification parameter to motivate and monitor the subject's participation in health wellness improvement programs.

### BACKGROUND

Health wellness systems generally refer to active processes that assist people in becoming aware of and improving conditions and lifestyle choices that affect their health and wellbeing. Health wellness focuses on awareness and proactive maintenance of physical, mental, spiritual and social wellbeing rather than focusing on disease treatment and management. The global wellness economy is a rapidly growing multi-trillion dollar range of interrelated businesses, products and activities, such as wellness evaluation and advice, exercise and personal training, meditation and psychology, spas and fitness centers, nutrition programs and supplements, wellness publications and blogs, wellness tourism, wellness real estate, and so forth.

Despite the growing interest in health wellness generally, conventional programs typically fail to take a proactive approach to assessing and improving men's testosterone levels and sex lives as part of the wellness management system. While much attention has been paid to medications for erectile disfunction, comparatively little has been paid to improving awareness and proactive maintenance of testosterone levels as an aspect of men's sexual contentment and wellness. Part of the problem is the highly subjective nature of questions about sexual contentment, the fact that most men have very little reliable information about what other men are experiencing in terms of sexual contentment, and the tendency of many men to exaggerate or become defensive when discussing the subject. There is, at present, a need for more effective ways to help men overcome these factors to improve testosterone levels, sexual contentment, and the perceived effects of aging on men's health and wellness.

### SUMMARY

The present invention meets the needs described above through a health wellness assessment and management system for men that computes the subject's “bio-wellness age” as a gamification parameter that takes into account the subject's chronological age and testosterone level, as well as a number of other evaluation factors, such as percent body fat, exercise habits, nutrition, smoking, and so forth. The system communicates the computed bio-wellness age to the subject, for example by display, print, email, text or other suitable mode of communication. The subject's bio-wellness age is continually recomputed as a way to motivate and monitor the subject's participation in health and wellness programs. While the system does not purport to accomplish any specific change to the subject's life span, expressing the subject's progress in accomplishing health and wellness program goals in the form of a bio-wellness age provides a simple and easy-to-understand “at-a-glance” gamification parameter used to monitor and motivate program compliance. The subject's bio-wellness age is also used to prescribe health and wellness program components, such as testosterone replacement therapy. The bio-wellness age is further used as a factor in the management of multiple health wellness system from a central location.

It will be understood that specific embodiments may include a variety of features in different combinations, as desired by different users. The specific techniques and systems for implementing particular embodiments of the invention and accomplishing the associated advantages will become apparent from the following detailed description of the embodiments and the appended drawings and claims.

### BRIEF DESCRIPTION OF THE FIGURES

The numerous advantages of the embodiments of the invention may be better understood with reference to the accompanying figures.

FIG. 1 is a block diagram of a health wellness assessment and management system.

FIG. 2 is a block diagram of a bio-wellness age module for the health wellness assessment and management system.

FIG. 3 is a table illustrating a body scan and appearance section of a representative bio-wellness age computation.

FIG. 4A is a table illustrating a first portion of a lifestyle and diet section of the representative bio-wellness age computation.

FIG. 4B is a table illustrating a second portion of the lifestyle and diet section of the representative bio-wellness age computation.

FIG. 5 is a table illustrating a vital signs and medical history section of the representative bio-wellness age computation.

FIG. 6 is a table illustrating a target testosterone section of the representative bio-wellness age computation.

FIG. 7 is a table illustrating a current bio-wellness age assessment section of the representative bio-wellness age computation.

FIG. 8 is a table illustrating a target bio-wellness age assessment section of the representative bio-wellness age computation.

FIG. 9 is a logic flow diagram of a bio-wellness program using bio-wellness age assessments.

FIG. 10 is a logic flow diagram for health wellness practice management system.

### DETAILED DESCRIPTION

The present invention meets the needs described above through a health wellness assessment and management system for men that computes a subject's “bio-wellness age” as a gamification parameter used to motivate and monitor the subject's progress in a health wellness improvement program. The subject's bio-wellness age is continually recomputed as he participates in the health wellness improvement plan as a way to monitor and motivate program participation. The subject's bio-wellness age takes into account his testosterone level, which brings the beneficial effects of testosterone replacement therapy into account in the bio-wellness age as a simple and easy-to-understand “at-a-glance” parameter. The subject's bio-wellness age is also used to prescribe health and wellness program components, such as testosterone replacement therapy. While the system does not purport to accomplish any specific change in the subject's live span, the bio-wellness age gamification parameter suggest that the subject “feels younger” by accomplishing the goals of the health wellness improvement plan.

The system computes the subject's “current bio-wellness age” based on his current testosterone level and a number of other current evaluation factors, such as percent body fat, exercise level, smoking, and so forth. The system guides a practitioner in developing a customized wellness improvement program for the subject including prescribed testosterone replacement therapy to bring the subject up to a target testosterone level, along with other targets for other evaluation factors, such as reduced percent body fat, increased exercise habits, improved nutrition, stopping smoking, and so forth. The system then computes the subject's “target bio-wellness age” assuming that the subject attains the target testosterone level by taking the prescribed testosterone replacement therapy, and also attains the other target evaluation factors through lifestyle changes. The subject's chronological age is compared to the current bio-wellness age and target bio-wellness age as a way to motivate the subject to participate in the wellness improvement program. For example, the system may show a chronological age of “56” and a current bio-wellness age of “67” along with a target bio-wellness age of “32” as an easy-to-understand, at-a-glance way to motivate the subject to engage in the wellness improvement program. Embodiments of the invention also include a health wellness practice management system that uses the bio-wellness age computations for multiple subjects, along with other elements of their bio-wellness assessments and customized health wellness plans, to manage multiple health wellness assessment and management systems from a central location.

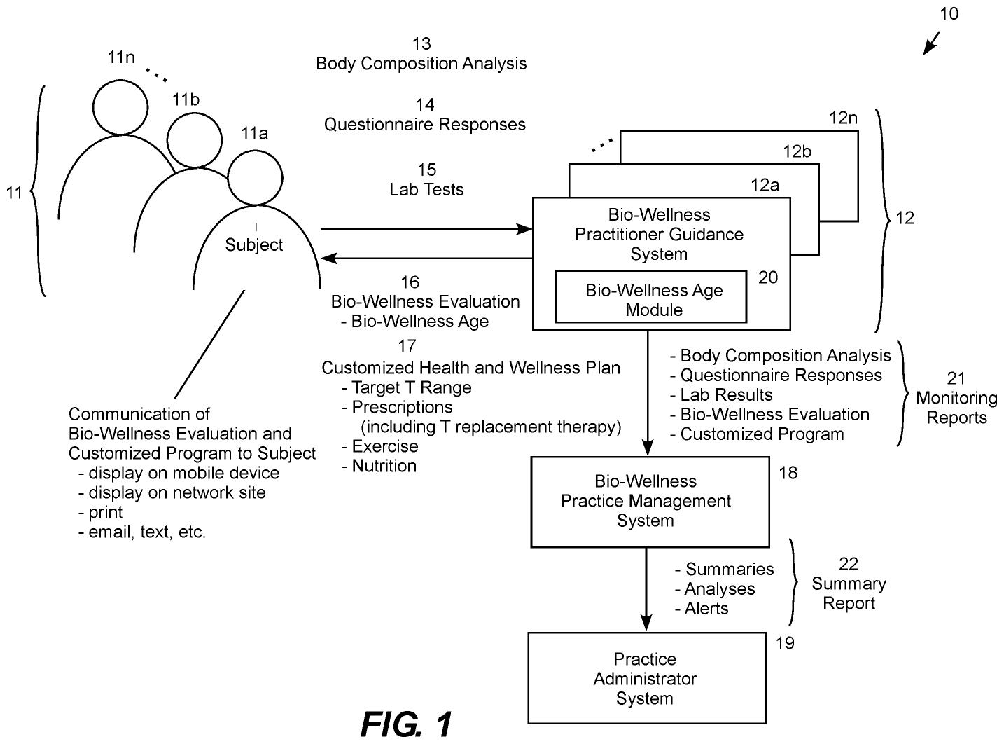

FIG. 1 is a block diagram of a health wellness assessment and management system 10. A subject 11 representing number of subjects 11a-n submits wellness evaluation information to a bio-wellness practitioner guidance system 12 representing number of bio-wellness practitioner guidance system 12a-n. For example, each bio-wellness practitioner guidance system may be used an individual bio-wellness practitioner to gather wellness evaluation information from a number of subjects. The bio-wellness practitioner is typically a medical doctor, physician's assistant, technical assistant or other practitioner trained and authorized to provide bio-wellness services including testosterone replacement therapy and related services. Each subject is typically a man interested in obtaining a bio-wellness evaluation and currently or potentially receiving these services. Each practitioner guidance system 12a-n is a computer-based, network-connected system including a processor and software running on the processor implementing associated functionality that computes the subject's current and target bio-wellness age as gamification parameters for encouraging the subject to participate in a health wellness plan and monitoring the subject's progress in accomplishing goals of the plan.

In this particular example, the evaluation information includes a body composition analysis 13, questionnaire responses 14, and lab test 15. The body composition analysis 13 may include results obtained from an InBody® or similar body analysis machine providing a body composition analysis including height, weight, percent body fat, and percent visceral body fat readings. The InBody body composition analysis system is a moderate-cost bioelectrical impedance machine considered appropriate for the bio-wellness evaluation. Other types of body composition analysis may be used, such as caliper measurements, hydrostatic weighing, air displacement plethysmography, dual-energy X-ray absorptiometry, and so forth.

The body composition analysis is typically augmented with vital signs and other relevant observations and measurements taken at the practitioner's location as part of the practitioner's interview, for example including blood pressure, heart rate, body temperature, waist circumference, joint flexibility and range of motion, muscular strength and endurance, respiration capacity, stress test performance, observed energy level, observed skin wrinkling, and so forth. The questionnaire responses 14 typically provide a range of health and wellness information gathered by the practitioner during an interview with the subject. The bio-wellness practitioner guidance system 12 provides a menu-driven system to assist the practitioner in conducting the interview and recording the subject's responses along with measurements, test results, observations, and other relevant information. In a particular embodiment, for example, the menu-driven questionnaire system 12 may prompt the practitioner to obtain and record subject's chronological age, medical history, family medical history, waistline measurement, appearance of facial wrinkles, sleep information, smoking information, alcohol consumption information, stress levels, exercise levels, diet and food quality information, sexual contentment information, general contentment information, and so forth.

The lab tests 15 typically provide the subject's cholesterol, vitamin-D and testosterone levels. While this information is generally considered sufficient for a typical bio-wellness evaluation, other lab information may be gathered. For example, other lab tests (e.g., blood sugar, liver function, uric acid, blood cell count, etc.) may be ordered based on the subject's medical history, family medical history and prescriptions.

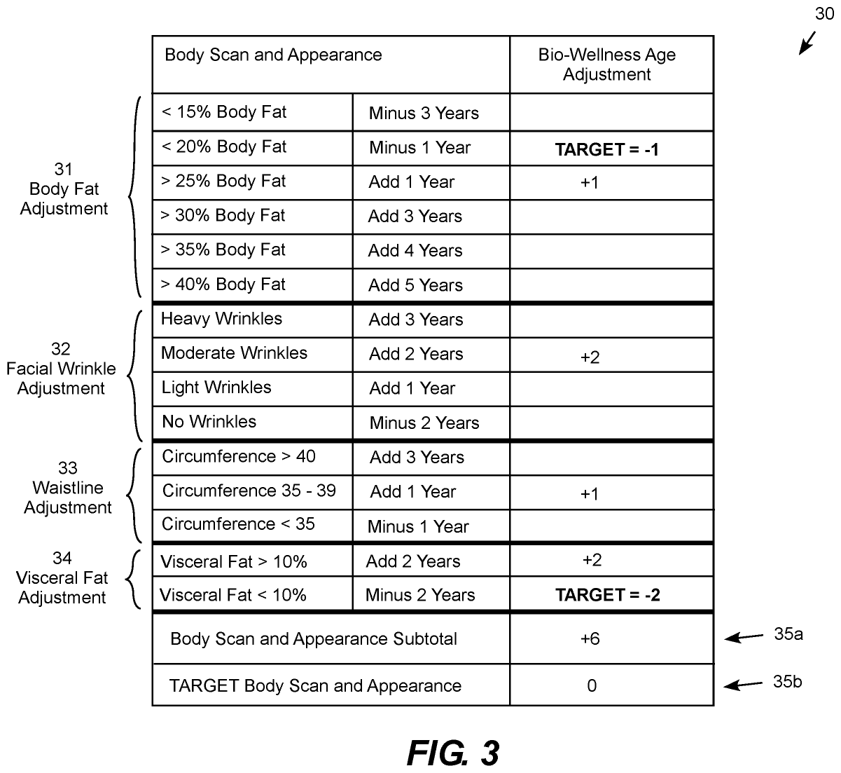

Using the bio-wellness practitioner guidance system 12, the practitioner provides the subject 11 with a bio-wellness evaluation 16, which includes a computed metric known as the subject's “current bio-wellness age.” If the subject already participating in a customized health wellness plan or otherwise interested in proceeding further, the practitioner also provides the subject with an updated or new customized wellness improvement plan 17 with specific prescriptions and line-item targets for certain evaluation factors, such as weight reduction, body fat reduction, and so forth. The system 12 is specifically designed to factor testosterone replacement therapy as well as attainment of an array of other evaluation factors in the computation of the subject's target bio-wellness age assuming that the subject attains a target testosterone level by taking the testosterone replacement therapy, and attains the other target evaluation factors through lifestyle changes. While the practitioner selects the specifics of the customized wellness improvement plan 17, the bio-wellness practitioner guidance system 12 guides the practitioner through the questionnaire 14 and other aspects of the bio-wellness assessment with a menu-driven system. FIGS. 3-8 illustrate a representative example of that menu-driven system for an example subject.

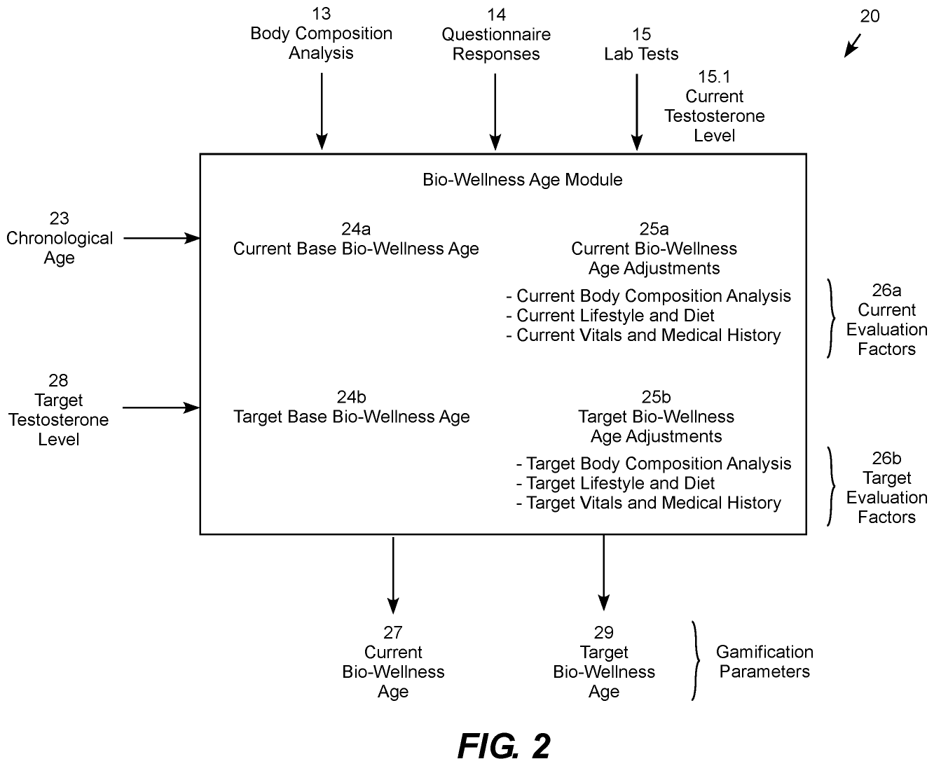

The practitioner guidance system 12 further guides the practitioner by computing the impact of the plan 17 on the subject's target bio-wellness age as described in more detail with reference to FIG. 2. The subject's chronological age is compared to the subject's current bio-wellness age and target bio-wellness age to provide an easy-to-understand, at-a-glance wellness gamification parameter to motivate the subject to engage and persist in the wellness improvement program. For example, the customized plan 17 may show a chronological age of “56” and a current bio-wellness age of “67” along with a target bio-wellness age of “32” as an easy-to-understand, at-a-glance way to motivate the subject to engage in a wellness improvement program designed to help the subject attain the target bio-wellness age.

The subject's bio-wellness age can also be recomputed at various points in the wellness improvement program as a gamification parameter to continually reinforce the motivation to continue with the program.

The health wellness assessment and management system 10 also includes a bio-wellness practice management system 18 that allows centralized monitoring, administration and control over a number of bio-wellness practitioner guidance system 12a-n. Each time one of the bio-wellness practitioner guidance system 12a-n conducts a bio-wellness evaluation 16 and/or produces or updates a customized plan 17, a monitoring report 21 is electronically delivered to the practice management system 18 identifying the practitioner, the subject, and salient information about the subject interaction, typically all of the information in the body composition analysis 13, questionnaire responses 14, lab results 15, the bio-wellness evaluation 16 and the customized plan 17 including the prescriptions and target testosterone level. The practice management system 18 compiles this information for multiple subjects and practitioners into a summary report 22, which is delivered to the practice administrator system 19, such as a specialized mobile phone app. The summary report 22 includes category summaries, analyses and alerts red-flagging any of the items raising potential concerns, such as target values or prescriptions that that may not be consistent with acceptable or typical ranges. The practice administrator system 19 allows the administrator to “drill down” to review the underlying data, down to the level of the actual form data collected from each subject and delivered to each subject by each practitioner stored on the practice management system 18. The practice administrator system 19 allows the administrator to contact the responsible practitioner, record interactions, issue instructions, disable the practitioner's practice management system 18, and take other response actions.

FIG. 2 is a block diagram of an example bio-wellness age module 20 for the health wellness assessment and management system 10. In this example, the bio-wellness age module 20 includes software that runs on the computer-based bio-wellness practitioner guidance system 12 that computes the subject's current and target bio-wellness age as gamification parameters for encouraging the subject to participate in the health wellness plan and monitoring the subject's progress accomplishing goals of the plan. The bio-wellness age module 20 receives information from the subject's body composition analysis 13, questionnaire responses 14, and lab tests 15 including the subject's current testosterone level 15.1. This includes a wide range of information considered relevant for the bio-wellness evaluation, such as the subject's chronological age 23, height, weight, percent body fat, percent visceral body fat, facial wrinkles, waistline measurement, sleep information, smoking information, alcohol consumption information, stress level, exercise information, food quality, sex life contentment, general contentment, vital signs, cholesterol, vitamin-D level, chronic disease information, and so forth. The practitioner typically enters this information into the bio-wellness age module 20 in preparation for and during an evaluation interview with the subject. Of course, some of these factors may be omitted and other factors may be considered as a matter of design choice. In more rigorous programs, for example, the evaluation may also include evaluations of joint flexibility and range of motion, muscular strength and endurance, respiration capacity, stress test performance, and so forth. As another example, specialized evaluations may be included for subjects in certain age groups, those with certain chronic diseases or conditions, those with certain medical or family medical histories, those undergoing physical therapy, those in aggressive weight loss programs, those in training for specific objectives, those participating in various types of recovery, and so forth.

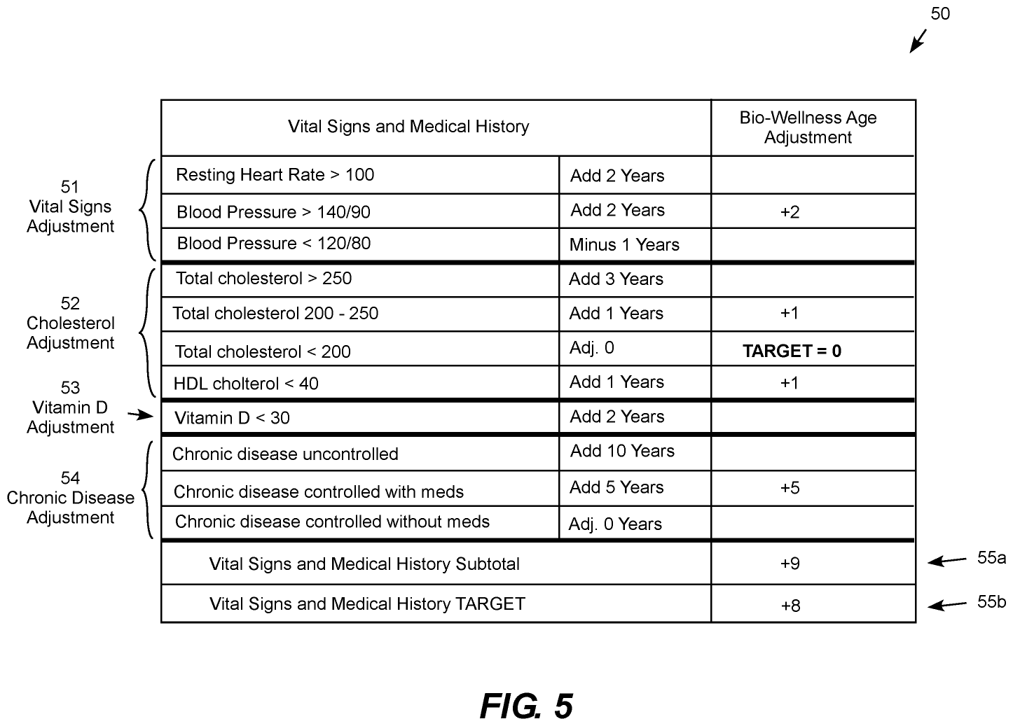

In this particular example, the bio-wellness age module 20 computes the subject's current base bio-wellness age 24a, which is based on the subject's current testosterone level 15.1 obtained from the lab tests 15. More specifically, the subject's current base bio-wellness age 24a is determined by comparing the subject's current testosterone 15.1 level to average testosterone levels for men in defined age categories, as described in more detail with reference to FIG. 6. The bio-wellness age module 20 also computes a number of current bio-wellness age adjustments 25a based on the subject's current evaluation factor's 26a. For example, a few years may be added for a high body fat percentage, a few years may be subtracted for good exercise, a few years may be added for smoking, and so forth. The subject's current bio-wellness age 27 is then computed by applying the current bio-wellness age adjustments 25a to the subject's current base bio-wellness age 24a. A specific illustrative example of this computation is shown in FIGS. 3-7. In one particular example, the bio-wellness evaluation shows a chronological age 23 of “56” and a current bio-wellness age 27 of “67” as a gamification parameter used to motivate the subject to initiate or continue with a wellness improvement program.

In this example, the practitioner also creates a customized wellness improvement plan 17 in part by entering a target testosterone level 28 along with prescribing testosterone replacement therapy designed to allow the subject to attain the target testosterone level. The bio-wellness age module 20 computes a target base bio-wellness age 24b assuming that the subject attains the target testosterone level 28 by taking the prescribed testosterone replacement therapy. The practitioner further creates the customized wellness improvement plan 17 by entering selected target evaluation factors 26b, such as reduced percent body fat, reduced weight, stopping smoking, improved diet, and so forth. The bio-wellness age module 20 computes target bio-wellness age adjustments 25b assuming that the subject attains the target evaluation factors 26b through lifestyle changes. FIGS. 3-8 also illustrate this computation for the representative example. In this example, the customized plan 17 shows a chronological age 23 of “56” and a current bio-wellness age 27 of “67” along with a target bio-wellness age 29 of “32” as easy-to-understand, at-a-glance gamification parameters used to monitor the subject's progress and motivate the subject to engage and continue in the wellness improvement program.

FIGS. 3-8 illustrate current and target bio-wellness age computations performed by the bio-wellness age module 20 for the representative example. The specific figures used in the example are merely illustrative and other factors, categories, line-items, age adjustments, etc. may be utilized as a matter of design choice. FIG. 3 is a table illustrating a “body scan and appearance” section 30 of a bio-wellness age computation for a representative subject. The “body fat adjustment” sub-category 31 includes several line-items for the practitioner to select regarding the subject's body fat including “<15% body fat,” “<20% body fat,” “>25% body fat,” “>30% body fat,” “>35% body fat” and “>40% body fat.” The bio-wellness age module 20 is preconfigured with a specific bio-wellness age adjustment for each line-item, specifically “minus 3 years” for “<15% body fat,” “minus 1 year” for “<20% body fat,” and so forth. Typically during an interview with the subject, the practitioner selects the corresponding line-item based on the subject's body composition analysis 13, which causes the bio-wellness age module 20 to automatically enter the corresponding age adjustment into the bio-wellness computation.

In this particular example, the practitioner has selected the “>25% body fat” line-item based on the subject's body composition analysis 13, which causes the bio-wellness age module 20 to enter “+1” into the current bio-wellness age adjustment for the body fat sub-category 31. This adds one year to the subject's current base bio-wellness age 24a in the computation of the subject's current bio-wellness age 27. In addition, the practitioner enters a target of “<20% body fat” line-item for the body fat adjustment sub-category 31, which causes the bio-wellness age module 20 to enter “−1” into the target bio-wellness age adjustment for the “body fat adjustment” sub-category 31 for the purpose of computing the subject's target bio-wellness age 29. This enters “reduce body fat to <20%” as a target in the subject's customized plan 17, and also subtracts one year from the subject's target base bio-wellness age 24b in the computation of the subject's target bio-wellness age 29.

Similarly, the “facial wrinkle adjustment” sub-category 32 includes line-items for “heavy wrinkles,” “moderate wrinkles,” “light wrinkles” and “no wrinkles.” The bio-wellness age module 20 is preconfigured with a specific bio-wellness age adjustment for each line-item, specifically “add 3 years” for “heavy wrinkles,” “add 2 years” for “moderate wrinkles,” “add one year” for “light wrinkles” and “minus 2 years” for “no wrinkles.” In this particular example, the practitioner has selected the “moderate wrinkles” line-item in the “facial wrinkle adjustment” sub-category 32 based on the practitioner's observation of the subject during the interview, which causes the bio-wellness age module 20 to enter “+2” into the current bio-wellness age adjustment for the “facial wrinkle adjustment” sub-category. This adds two years to the subject's current base bio-wellness age 24a in the computation of the subject's current bio-wellness age 27. The practitioner does not enter a target for this line-item.

In addition, the “waistline adjustment” sub-category 33 includes line-items for “circumference >40,” “circumference 35-39” and “circumference <35.” The bio-wellness age module 20 is preconfigured with a specific bio-wellness age adjustment for each line-item, specifically “add 3 years” for “circumference >40,” “add 1 year” for “circumference 35-39,” and “minus 1 year” for “circumference <35.” In this particular example, the practitioner has selected the “circumference 35-39” line-item in the “waistline adjustment” sub-category 33 based on the practitioner's measurement of the subject's waistline during the interview, which causes the bio-wellness age module 20 to enter “+1” into the current bio-wellness age adjustment for the “waistline adjustment” sub-category 33. This adds one year to the subject's current base bio-wellness age 24a in the computation of the subject's current bio-wellness age 27. The practitioner does not enter a target for this line-item.

In addition, the “visceral fat adjustment” sub-category 34 includes line-items for “visceral fat >10%” and “visceral fat <10%.” The bio-wellness age module 20 is preconfigured with a specific bio-wellness age adjustment for each line-item, specifically “add 2 years” for “visceral fat >10%” and “minus 2 years” for “visceral fat <10%.” In this particular example, the practitioner selected the “visceral fat >10%” line-item for the “visceral fat adjustment” sub-category 34 based on the subject's body composition analysis 13, which causes the bio-wellness age module 20 to enter “+2” into the bio-wellness age adjustment for the “visceral fat adjustment” sub-category for the purpose of computing the subject's current bio-wellness age 27. This adds two years to the subject's current base bio-wellness age 24a in the computation of the subject's current bio-wellness age 27. In addition, the practitioner has selected a target of “<10% visceral fat” line-item for the “visceral fat adjustment” sub-category 34, which causes the bio-wellness age module 20 to enter “−2” into the bio-wellness age adjustment for the “visceral fat adjustment” sub-category 34 for the purpose of computing the subject's target bio-wellness age. This subtracts two years from the subject's target base bio-wellness age 24b in the computation of the subject's target bio-wellness age 29.

At the bottom of FIG. 3, the bio-wellness age module 20 totals the “current body scan and appearance” adjustment factors 31-34, resulting in a “body scan and appearance” current subtotal adjustment 35a of “+6” added to the subject's base bio-wellness age 24 in the computation of the subject's current bio-wellness age 27. Similarly, the bio-wellness age module 20 also computes a “target body scan and appearance” subtotal adjustment 35b of “0” which is added to the subject's target base bio-wellness age 24b in the computation of the subject's target bio-wellness age 29.

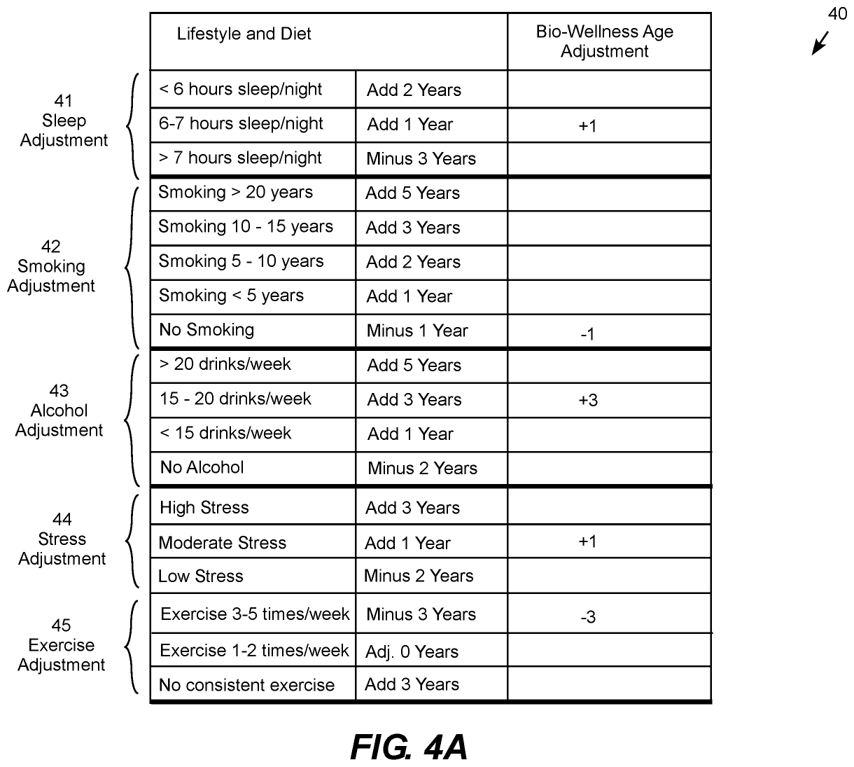

FIG. 4A is a table illustrating a first portion of a “lifestyle and diet” section 40 of the representative bio-wellness age computation. The “sleep adjustment” sub-category 41 includes several line-items for the practitioner to select regarding the subject's sleep habits including “<6 hours per night,” “6-7 hours per night,” and “>7 hours per night.” The bio-wellness age module 20 is preconfigured with a specific bio-wellness age adjustment for each line-item, specifically “add 2 years” for “<6 hours per night,” “add 1 year” for “6-7 hours per night,” and “minus 3 years” for “>7 hours per night.” In this particular example, the practitioner has selected the “6-7 hours sleep per night” line-item in the “sleep adjustment” sub-category 41 based on the practitioner's interview with the subject, which causes the bio-wellness age module 20 to enter “+1” into the bio-wellness age adjustment for the “sleep adjustment” sub-category. This adds one year to the subject's current base bio-wellness age 24a in the computation of the subject's current bio-wellness age 27. The practitioner does not enter a target for this line-item.

Similarly, the “smoking adjustment” sub-category 42 includes line-items for “smoking >20 years,” “smoking 10-15 years,” “smoking 5-10 years,” “smoking <5 years” and “no smoking.” The bio-wellness age module 20 is preconfigured with a specific bio-wellness age adjustment for each line-item, specifically “add 5 years” for “smoking >20 years,” “add 3 years” for “smoking 10-15 years,” “add 2 years” for “smoking 5-10 years” and so forth. In this particular example, the practitioner has selected the “no smoking” line-item in the “smoking adjustment” sub-category 42, which causes the bio-wellness age module 20 to enter “−1” into the bio-wellness age adjustment for the “smoking adjustment” sub-category 42 based on the practitioner's interview with the subject. This subtracts one year from the subject's current base bio-wellness age 24a in the computation of the subject's current bio-wellness age 27. The practitioner does not enter a target for this line-item.

In addition, the “alcohol adjustment” sub-category 43 includes line-items for “>20 drinks per week,” “15-20 drinks per week,” “<15 drinks per week” and “no drinking.” The bio-wellness age module 20 is preconfigured with a specific bio-wellness age adjustment for each line-item, specifically “add 5 years” for “>20 drinks per week,” “add 3 years” for “15-20 drinks per week,” and so forth. In this particular example, the practitioner has selected the “15-20 drinks per week” line-item in the “alcohol adjustment” sub-category 43 based on the practitioner's interview with the subject, which causes the bio-wellness age module 20 to enter “+3” into the bio-wellness age adjustment for the “alcohol adjustment” sub-category 43. This adds three years to the subject's current base bio-wellness age 24a in the computation of the subject's current bio-wellness age 27. The practitioner does not enter a target for this line-item.

In addition, the “stress adjustment” sub-category 44 includes line-items for “high stress,” “moderate stress,” and “low stress.” The bio-wellness age module 20 is preconfigured with a specific bio-wellness age adjustment for each line-item, specifically “add 3 years” for “high stress,” “add 1 year” for “moderate stress” and “minus 1 year” for “low stress.” In this particular example, the practitioner has selected the “moderate stress” line-item in the “stress adjustment” sub-category 44 based on the practitioner's interview with the subject, which causes the bio-wellness age module 20 to enter “+1” into the bio-wellness age adjustment for the “stress adjustment” sub-category 44. This adds one year to the subject's current base bio-wellness age 24a in the computation of the subject's current bio-wellness age 27. The practitioner does not enter a target for this line-item.

In addition, the “exercise adjustment” sub-category 45 includes line-items for “exercise 3-5 times per week,” “exercise 1-2 times per week,” and “no consistent exercise.” The bio-wellness age module 20 is preconfigured with a specific bio-wellness age adjustment for each line-item, specifically “minus 3 years” for “exercise 3-5 times per week,” “adjust 0 years” for “exercise 1-2 times per week” and “add 3 years” for “no consistent exercise.” In this particular example, the practitioner has selected the “exercise 3-5 times per week” line-item in the “exercise adjustment” sub-category 45 based on the practitioner's interview with the subject, which causes the bio-wellness age module 20 to enter “-3” into the bio-wellness age adjustment for the “exercise adjustment” sub-category 45. This subtracts three years from the subject's current base bio-wellness age 24a in the computation of the subject's current bio-wellness age 27. The practitioner does not enter a target for this line-item.

FIG. 4B is a table illustrating a continuation of the lifestyle and diet section 40 of the bio-wellness age computation. The “food quality adjustment” sub-category 46 includes line-items for “poor quality meals >7 meals per week,” “poor quality meals 4-7 meals per week,” “poor quality meals <4 meals per week,” “fruits and vegetables >5 servings per day” and “beans, nuts and seeds in weekly diet.” The bio-wellness age module 20 is preconfigured with a specific bio-wellness age adjustment for each line-item, specifically “add 3 years” for “poor quality meals >7 meals per week,” “add 1 year” for “poor quality meals 4-7 meals per week,” “minus 2 years” for “poor quality meals <4 meals per week” and so forth. In this particular example, the practitioner has selected of “poor quality meals 4 to 7 times per week” line-item for the “food quality adjustment” sub-category 46 based on the practitioner's interview with the subject, which causes the bio-wellness age module 20 to enter “+1” into the bio-wellness age adjustment for the “food quality adjustment” sub-category 46 for the purpose of computing the subject's current bio-wellness age 27. This subtracts three years from the subject's current base bio-wellness age 24a in the computation of the subject's current bio-wellness age 27. In addition, the practitioner enters a target of “poor quality meals <4 meals per week” line-item for the “food quality adjustment” sub-category 46. This causes the bio-wellness age module 20 to enter “−2” into the bio-wellness age adjustment for the “food quality adjustment” sub-category 46, which subtracts two years from the subject's target base bio-wellness age 24b in the computation of the subject's target bio-wellness age 29.

In addition, the “sex life adjustment” sub-category 47 includes line-items for “happy active sex life” and “inactive or difficulties in sex life.” The bio-wellness age module 20 is preconfigured with a specific bio-wellness age adjustment for each line-item, specifically “minus 3 years” for “happy active sex life” and “add 3 years” for “inactive or difficulties in sex life.” In this particular example, the practitioner selected the “happy active sex life” line-item in the “sex life adjustment” sub-category 47 based on the practitioner's interview with the subject, which causes the bio-wellness age module 20 to enter “−5” into the bio-wellness age adjustment for the “sex life adjustment” sub-category 47. This subtracts five years from the subject's current base bio-wellness age 24a in the computation of the subject's current bio-wellness age 27. The practitioner does not enter a target for this line-item.

In addition, the “contentment adjustment” sub-category 48 includes line-items for “happy active lifestyle” and “unhappy or sedentary lifestyle.” The bio-wellness age module 20 is preconfigured with a specific bio-wellness age adjustment for each line-item, specifically “minus 3 years” for “happy active lifestyle” and “add 3 years” for “unhappy or sedentary lifestyle.” In this particular example, the practitioner selected the “happy active lifestyle” line-item in the “lifestyle adjustment” sub-category 48, which causes the bio-wellness age module 20 to enter “−3” into the bio-wellness age adjustment for the “lifestyle adjustment” sub-category 48 based on the practitioner's interview with the subject, which causes the bio-wellness age module 20 to enter “−3” into the bio-wellness age adjustment for the “contentment adjustment” sub-category 48. This subtracts three years from the subject's current base bio-wellness age 24a in the computation of the subject's current bio-wellness age 27. The practitioner does not enter a target for this line-item.

At the bottom of FIG. 4B, the bio-wellness age module 20 totals the “lifestyle and diet” adjustment factors 41-48, resulting in a “lifestyle and diet subtotal” adjustment 49a of “−8” subtracted from the subject's current base bio-wellness age 24a in the computation of the subject's current bio-wellness age 27. Similarly, the bio-wellness age module 20 computes a “TARGET lifestyle and diet” subtotal adjustment 49b of “−11” which is subtracted from the subject's target base bio-wellness age 24b in the computation of the subject's target bio-wellness age 29.

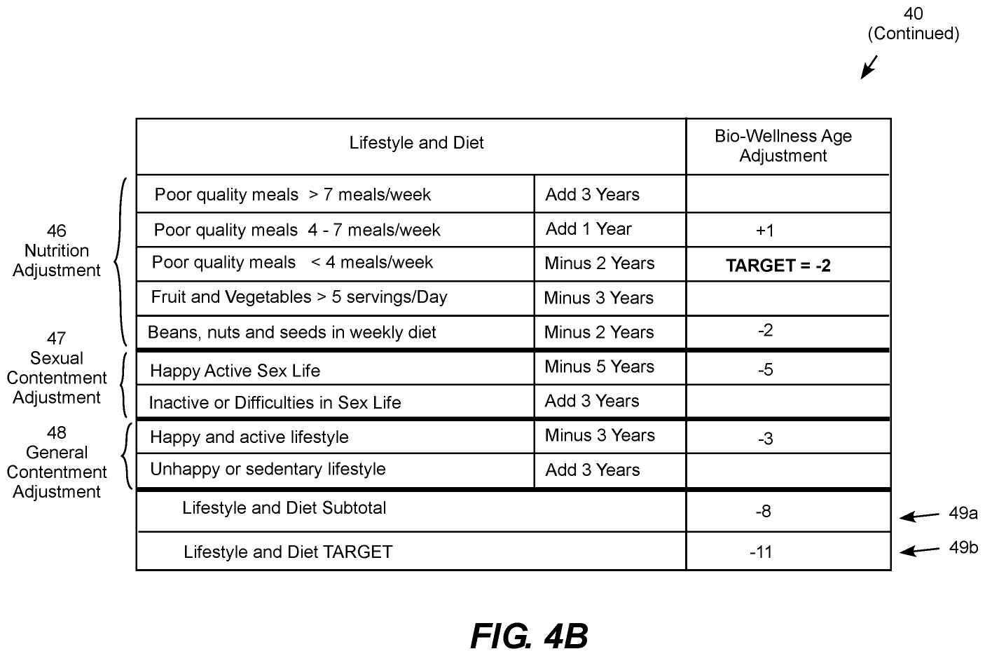

FIG. 5 is a table illustrating a “vital signs and medical history” section 50 of the representative bio-wellness age computation. The “vital signs adjustment” category 51 includes several line items for the practitioner to select including “resting heart rate >100,” “blood pressure >140/90” and “blood pressure <120/80.” The bio-wellness age module 20 is preconfigured with a specific bio-wellness age adjustment for each line-item, specifically “add 2 years” for “resting heart rate >100,” “add 2 years” for “blood pressure >140/90” and “minus 1 year” for “blood pressure <120/80.” In this particular example, the practitioner has selected the “blood pressure >140/90” line item based on a blood pressure measure taken during the practitioner's interview with the subject, which causes the bio-wellness age module 20 to enter “+2” into the bio-wellness age adjustment for the “vital signs adjustment” sub-category 51. This adds two years to the subject's current base bio-wellness age 24a in the computation of the subject's current bio-wellness age 27. The practitioner does not enter a target for this line-item.

The “cholesterol adjustment” category 52 includes several line items for the practitioner to select including “total cholesterol >250,” “total cholesterol 200 to 250,” “total cholesterol <200” and “HDL cholesterol <40.” The bio-wellness age module 20 is preconfigured with a specific bio-wellness age adjustment for each line-item, specifically “add 3 years” for “total cholesterol >250,” “add 1 year” for “total cholesterol 200 to 250,” “adjust 0 years” for “total cholesterol <200” and “add 1 year” for “HDL cholesterol <40.” In this particular example, the practitioner has selected the “total cholesterol 200 to 250” line item based on the subject's lab tests 15, which causes the bio-wellness age module 20 to enter “+1” into the bio-wellness age adjustment for the “cholesterol adjustment” sub-category 52. The practitioner has also selected the “HDL cholesterol <40” line item based on the subject's lab tests 15, which causes the bio-wellness age module 20 to enter another “+1” into the bio-wellness age adjustment for the “cholesterol adjustment” sub-category 52. This adds two years to the subject's current base bio-wellness age 24a in the computation of the subject's current bio-wellness age 27. In addition, the practitioner enters a target of “total cholesterol <200” line-item for the “cholesterol adjustment” sub-category 52. This causes the bio-wellness age module 20 to enter “0” into the bio-wellness age adjustment for the “cholesterol adjustment” sub-category 52 for the purpose of computing the subject's target bio-wellness age 29.

The “vitamin-D adjustment” category 53 includes one line-item for “vitamin-D <30” with a bio-wellness age adjustment of “add 2 years”. The practitioner did not enter an adjustment factor here based on the subject's lab results 15.

The “chronic disease adjustment” category 54 includes several line items for the practitioner to select including “chronic disease uncontrolled,” “chronic disease controlled with meds” and “chronic disease controlled without meds.” The bio-wellness age module 20 is preconfigured with a specific bio-wellness age adjustment for each line-item, specifically “add 10 years” for “chronic disease uncontrolled,” “add 5 year2” for “chronic disease controlled with meds” and “adjust 0 years” for “chronic disease controlled without meds.” In this particular example, the practitioner has selected the “chronic disease controlled with meds” line item based on the practitioner's interview with the subject, which causes the bio-wellness age module 20 to enter “+5” into the bio-wellness age adjustment for the “chronic disease adjustment” sub-category 54. This adds five years to the subject's current base bio-wellness age 24a in the computation of the subject's current bio-wellness age 27.

At the bottom of FIG. 5, the bio-wellness age module 20 totals the “vital signs and medical history” adjustment factors 51-54, resulting in a “vital signs and medical history subtotal” adjustment 55a of “+9” added to the subject's current base bio-wellness age 24a in the computation of the subject's current bio-wellness age 27. Similarly, the bio-wellness age module 20 also computes a “TARGET” vital signs and medical history “subtotal” adjustment 55b of “+8” which is added to the subject's target base bio-wellness age 24b in the computation of the subject's target bio-wellness age 29.

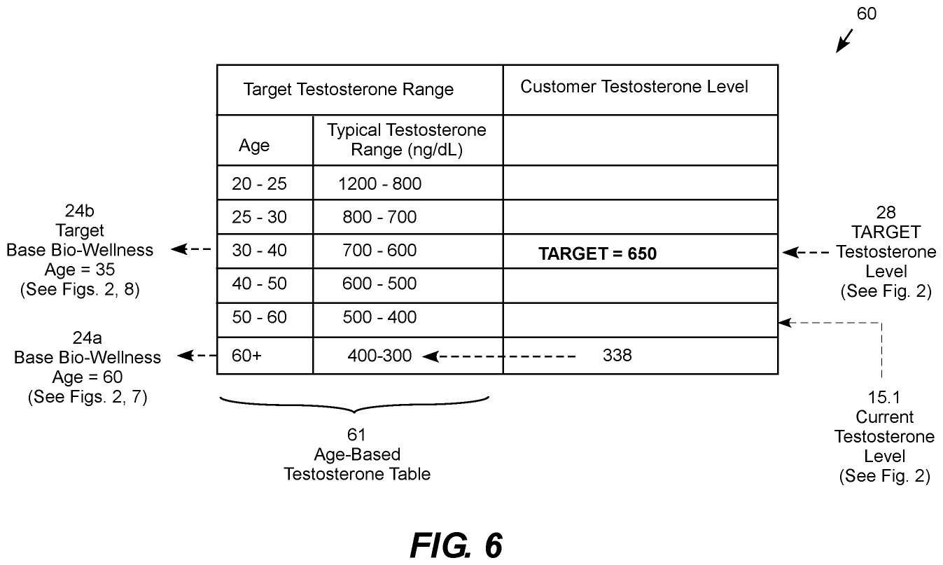

FIG. 6 is a table illustrating a target testosterone section 60 of the bio-wellness age computation. The section includes an age-based testosterone table 61 showing age ranges for men and a typical testosterone range for each age range. For example, the table 61 shows a typical testosterone range in nanograms per decilitre (ng/dL) of 1200-800 for the age range of 20-25, a typical testosterone range of 800-700 for the age range of 25-30, a typical testosterone range of 700-600 for the age range of 30-40, a typical testosterone range of 600-500 for the age range of 40-50, a typical testosterone range of 500-400 for the age range of 50-60, and a typical testosterone range of 400-300 for the age range of 60 and above. The bio-wellness age module 20 determines the subject's current base bio-wellness age 24a by identifying the age group for the typical testosterone range in the table 61 corresponding to the subject's current testosterone range 15.1 from the subject's lab tests 15. In the particular example shown in FIG. 6, the subject's current testosterone level 15.1 is 338 ng/dL obtained from the subject's lab tests 15. This corresponds to the typical testosterone range 400-300 in the table 61, which in turn corresponds to the age range of 60 and above. The bio-wellness age module 20 uses this information to set the subject's current base bio-wellness age 24a to age 60, which is further used in the bio-wellness assessment in the manner as shown in FIGS. 2 and 7.

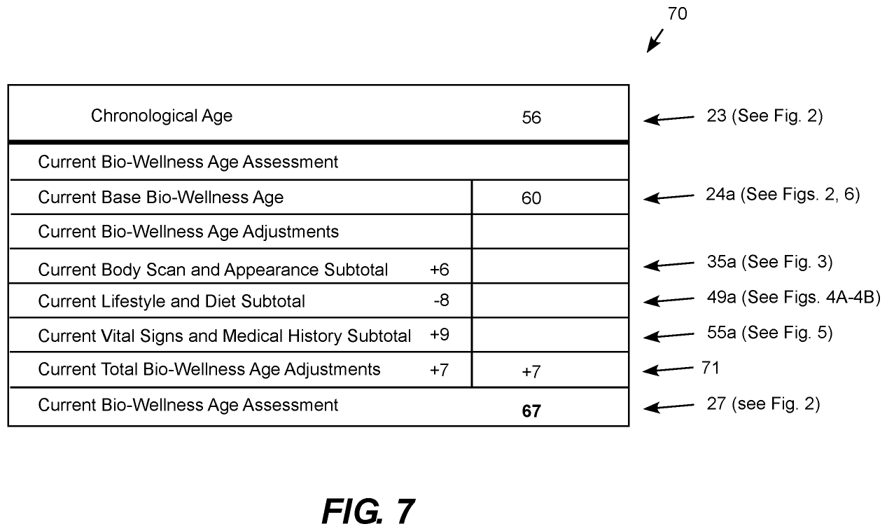

Similarly, the bio-wellness age module 20 determines the subject's target base bio-wellness age 24b based on the target testosterone level 28 selected by the practitioner as part of the customized plan 17 for the subject. The module 20 identifies the age group for the typical testosterone range in the table 61 corresponding to the subject's target testosterone range 28 from the subject's customized plan 17. In the particular example shown in FIG. 6, the subject's target testosterone range 28 is 650ng/dL. This corresponds to the typical testosterone range 700-600 in the table 61, which in turn corresponds to the age range of 30-40. The bio-wellness age module 20 uses this information to set the subject's target base bio-wellness age 24b to age 35, which is further used in the bio-wellness assessment in the manner as shown in FIGS. 2 and 8.

FIG. 7 is a table illustrating a current bio-wellness age assessment section 70 of the bio-wellness age computed by the bio-wellness age module 20. The subject's chronological age 23 as shown in FIG. 2 (age 56 in the specific example shown) is included for reference. The bio-wellness age assessment begins with the subject's current base bio-wellness age 24a as shown in FIGS. 2 and 6 (age 60 in the specific example shown). The current base bio-wellness age 24a is then adjusted by the current body scan and appearance subtotal 35a as shown in FIG. 3 (+6 years in the specific example shown), the current body scan an appearance subtotal 49a as shown in FIGS. 4A-4B (−8 years in the specific example shown), and the current vital signs and medical history subtotal 55a as shown in FIG. 5 (+9 years in the specific example shown). The sum of these subtotals is the current total bio-wellness age adjustment 71 (+7 years in the specific example shown). Adding this figure (+7 years in the specific example shown) to the subject's current base bio-wellness age 24a (age 60 in the specific example shown) produces the subject's current bio-wellness age assessment 27 as shown in FIG. 2 (age 67 in the specific example shown).

FIG. 8 is a table illustrating a target bio-wellness age assessment section 80 of the bio-wellness age computed by the bio-wellness age module 20. The subject's chronological age 23 as shown in FIG. 2 (age 56 in the specific example shown) is included for reference. The bio-wellness age assessment begins with the subject's target base bio-wellness age 24b as shown in FIGS. 2 and 6 (age 35 in the specific example shown). The target base bio-wellness age 24b is then adjusted by the target body scan and appearance subtotal 35b as shown in FIG. 3 (+0 years in the specific example shown), the target body scan an appearance subtotal 49b as shown in FIGS. 4A-4B (−11 years in the specific example shown), and the target vital signs and medical history subtotal 55b as shown in FIG. 5 (+8 years in the specific example shown). The sum of these subtotals is the current total bio-wellness age adjustment 81 (−3 years in the specific example shown). Adding this figure (−3 years in the specific example shown) to the subject's target base bio-wellness age 24b (age 35 in the specific example shown) produces the subject's target bio-wellness age assessment 29 as shown in FIG. 2 (age 32 in the specific example shown).

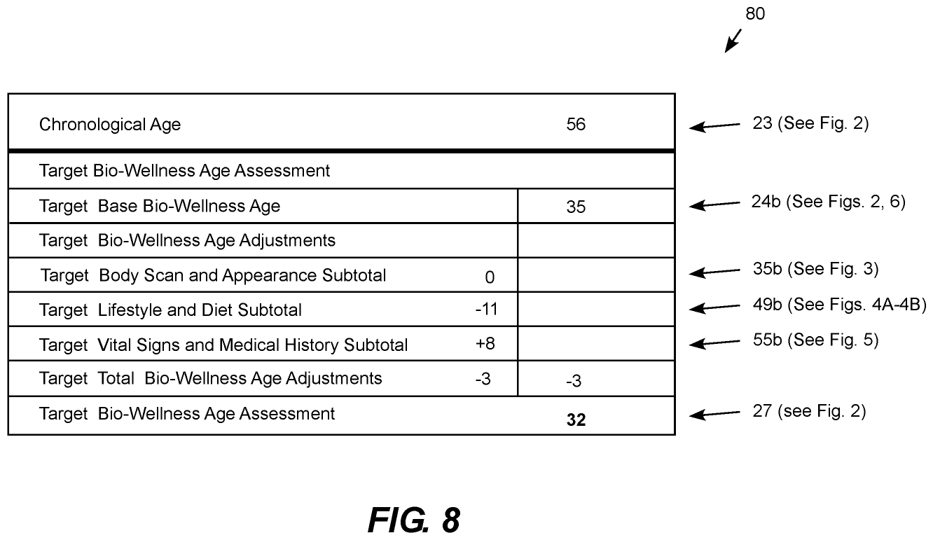

FIG. 9 is a logic flow diagram 90 for a bio-wellness program using bio-wellness age assessments. Referring also to FIGS. 1 and 2, in step 91 a practitioner gathers health wellness information from a subject 11 including a body composition analysis 13, questionnaire responses 14, and lab results 15 including the subject's current testosterone measurement 15.1. The practitioner enters this information into a bio-wellness practitioner guidance system 12. Step 91 is followed by step 92, in which a bio-wellness age module 20 of the system 12 computes the subject's current bio-wellness age 27 based on the subject's current base bio-wellness age 24a, which is based on the subject's current testosterone measurement 15.1, and a number of current bio-wellness age adjustments 25a based on a number of current evaluation factors 26a. FIGS. 3-5 show an example of the current bio-wellness age adjustments 25a and current evaluation factors 26a, while FIG. 6 shows an example determination of the subject's current base bio-wellness age 24a, and FIG. 7 shows an example computation of the subject's current bio-wellness age 27.

Step 92 is followed by step 93, in which the practitioner enters a customized bio-wellness plan 17 for the subject into the bio-wellness practitioner guidance system 12. The customized plan 17 typically includes a target testosterone level 28 and a prescription for testosterone replacement therapy designed to cause the subject to attain the target testosterone level. The customized plan 17 may also include one or more additional target evaluation factors 26b, which the subject should pursue through lifestyle changes. Step 93 is followed by step 94, in which the bio-wellness age module 20 computes the subject's target bio-wellness age 29 based on the subject's target base bio-wellness age 24b, which is based on the subject's target testosterone level 28. The subject's target base bio-wellness age 24b may also be adjusted by one or more target bio-wellness age adjustments 25b, which are based on the target evaluation factors 26b. Step 94 is followed by step 95, in which routine 90 may be repeated to assess the subject's progress in achieving the goals of the health wellness program.

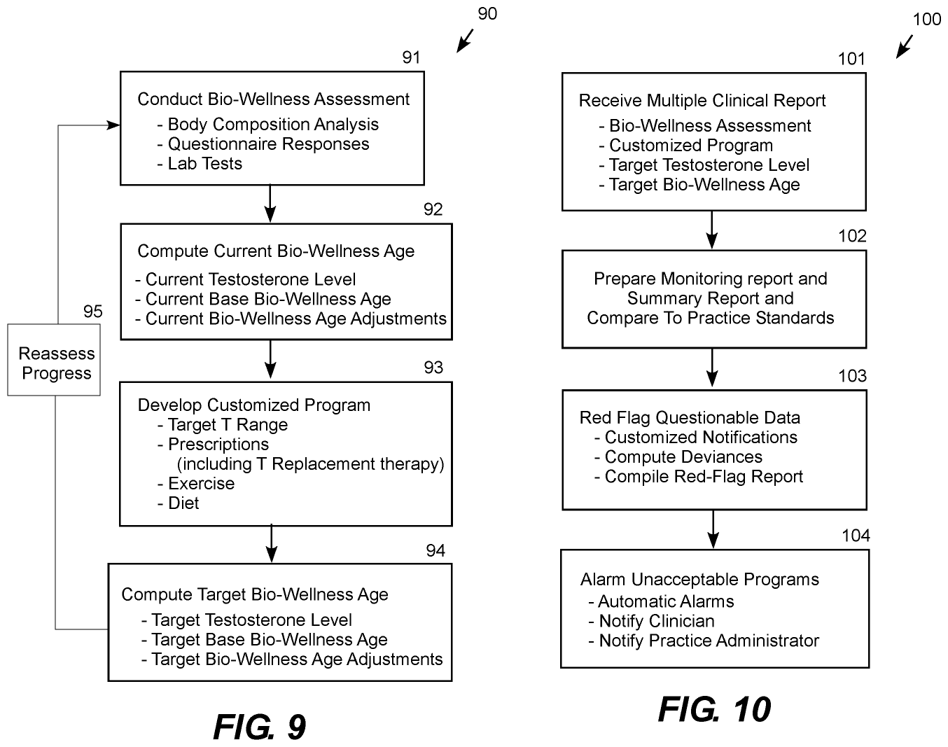

FIG. 10 is a logic flow diagram 100 for managing a health wellness practice. Referring also to FIG. 1, in step 101 a bio-wellness practice management system 18 receives a monitoring report 21 from a bio-wellness practitioner guidance systems 12. The monitoring report typically includes all of the information from the body composition analysis 13, questionnaire responses 14, lab tests 15, bio-wellness evaluation 16, and the customized plan 17 for each participating practitioner and subject. The bio-wellness practice management system 18 also stores this information later access as well as multi-subject and multi-practitioner analysis. Step 101 is followed by step 102, in which the bio-wellness practice management system 18 generates a summary report 22 based on this information with summary level information, analyses and alerts. Step 102 is followed by step 103, in which the bio-wellness practice management system 18 detects and red-flags questionable data, which typically includes customizable notifications, computed deviances, and a red-flag report. For example, target testosterone levels that are considered overly conservative or overly aggressive may be red-flagged for administrative review.

Step 103 is followed by step 104, in which the bio-wellness practice management system 18 sends the summary report 22 to a practice administrator system 19, where the red flags activate alerts, such as automatic alarms, clinician notifications, and practice administrator notifications. The summary report 22 including alerts may be transmitted, for example, by email or text, to a practice administrator system 19.

The present disclosure may be implemented using a cloud-based system that operates in concert with apps running on one or more mobile communication devices (e.g., smartphones) and one or more additional network-based or cloud-based platforms. Each of these devices includes a controller utilizing a general purpose computing device, such as a microprocessor controlled by specialized computer software. As such, embodiments of the disclosure may comprise adapting or reconfiguring presently existing equipment. Alternatively, original equipment may be provided embodying the disclosure.

All of the methods described in this disclosure may include storing non-transient computer-executable instructions and associated results in a non-transient storage medium. These computer-executable instructions and results may include any of the computer-implemented procedures or results described in this disclosure and may be stored in any manner known in the art. The storage medium may include any storage medium described in this disclosure or any other suitable storage medium known in the art. After the computer-executable instructions or results have been stored, they can be accessed in the storage medium and used by any of the method or system embodiments described in this disclosure, formatted for display to a user, used by another software module, method, or system, etc. Furthermore, the results may be stored “permanently,” “semi-permanently,” temporarily, or for some period of time. For example, the storage medium may be random access memory (RAM), and the results may not necessarily persist indefinitely in the storage medium.

Those having skill in the art will appreciate that there are various vehicles by which processes and/or systems and/or other technologies described in this disclosure can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described in this disclosure may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

Those skilled in the art will recognize that it is common within the art to describe devices and/or processes in the fashion set forth in this disclosure, and then use engineering practices to integrate such described devices and/or processes into data processing systems. That is, at least a portion of the devices and/or processes described can be integrated into a data processing system via a reasonable amount of experimentation. Those having skill in the art will recognize that a typical data processing system generally comprises one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems. All of the technology described in this disclosure is suitable for implementation using commercially available computing devices, such as microprocessors executing computer-executable software. These computing devices may be interconnected via the Internet, mobile telephone voice and data system, or other data suitable network.

This disclosure sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components may be combined to achieve a particular functionality can be seen as “associated with” each other such that the desired functionality is achieved, irrespective of architectures or intermediate components. Likewise, any two components so associated can also be viewed as being “connected”, or “coupled”, to each other to achieve the desired functionality, and any two components capable of being so associated can also be viewed as being “functionally connected” to each other to achieve the desired functionality. Specific examples of functional connection include but are not limited to physical connections and/or physically interacting components and/or wirelessly communicating and/or wirelessly interacting components and/or logically interacting and/or logically interacting components.

While particular aspects of the present subject matter have been shown and described in detail, it will be apparent to those skilled in the art that, based upon the teachings of this disclosure, changes and modifications may be made without departing from the subject matter described in this disclosure and its broader aspects and, therefore, the appended claims are to encompass within their scope all such changes and modifications as are within the true spirit and scope of the subject matter described in this disclosure. Although particular embodiments of this disclosure have been illustrated, it is apparent that various modifications and embodiments of the disclosure may be made by those skilled in the art without departing from the scope and spirit of the disclosure.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. The disclosure is defined by the following claims, which should be construed to encompass one or more structures or function of one or more of the illustrative embodiments described above, equivalents and obvious variations. it will therefore be appreciated that present invention provides significant improvements in electric power circuit reclosers. The foregoing relates only to the exemplary embodiments of the present invention, and that numerous changes may be made therein without departing from the spirit and scope of the invention as defined by the following claims.

The invention claimed is:

1. A health wellness assessment and management system configured to perform a method comprising:
receiving a current body composition analysis for a subject;
receiving a current lab test including a testosterone measurement for the subject;
receiving a current questionnaire response including a chronological age for the subject;
computing a current bio-wellness age for the subject based on the current body composition analysis, the current lab test, and the current questionnaire response;
communicating the current bio-wellness age to the subject as a gamification parameter to motivate and monitor the subject's participation in health wellness improvement programs.

2. The system of claim 1, further configured for:
generating a customized health and wellness plan including a target testosterone level and a target bio-wellness age based on the target testosterone level for the subject;
communicating the customized health and wellness plan to the subject.

3. The system of claim 2, further configured for:
generating a testosterone replacement therapy selected to cause the subject to attain the target testosterone level;
communicating the testosterone replacement therapy to the subject.

4. The system of claim 2, further configured for:
receiving a subsequent body composition analysis for the subject;
receiving a subsequent testosterone measurement for the subject;
computing a subsequent bio-wellness age for the subject based on the subsequent body composition analysis and the testosterone measurement;
communicating the subsequent bio-wellness age to the subject as a subsequent gamification parameter to motivate and monitor the subject's participation in health wellness improvement programs.

5. The system of claim 2, further configured for:
receiving another current questionnaire response including current exercise information for the subject and computing the current bio-wellness age based on the current exercise information for the subject;
generating target exercise information for the subject and computing the target bio-wellness age based on the target exercise information for the subject.

6. The system of claim 2, further configured for:
receiving another current questionnaire response including current nutrition information for the subject and computing the current bio-wellness age based on the current nutrition information for the subject;
generating target nutrition information for the subject and computing the target bio-wellness age based on the target nutrition information for the subject.

7. The system of claim 2, further configured for:
receiving another current questionnaire response including current sexual contentment information for the subject and computing the current bio-wellness age based on the current sexual contentment information for the subject;
generating target sexual contentment information for the subject and computing the target bio-wellness age based on the target sexual contentment information for the subject.

8. The system of claim 2, further configured for:
receiving current body fat information for the subject and computing the current bio-wellness age based on the current body fat information for the subject;
generating target body fat information for the subject and computing the target bio-wellness age based on the target body fat information for the subject.

9. The system of claim 2, further configured for:
receiving current visceral body fat information for the subject and computing the current bio-wellness age based on the current body fat information for the subject;
generating target visceral body fat information for the subject and computing the target bio-wellness age based on the target body fat information for the subject.

10. The system of claim 1, further configured for computing the current bio-wellness age based on the testosterone level for the subject and typical testosterone level for age-based categories of men.

11. The system of claim 10, further configured for computing the current bio-wellness age based on the testosterone level for the subject and typical testosterone level for age-based categories of men adjusted by one or more of the following:
body fat adjustment;
facial wrinkle adjustment;
waistline adjustment;
visceral fat adjustment;
sleep adjustment;
smoking adjustment;
alcohol adjustment;
stress adjustment;
exercise adjustment;
nutrition adjustment;
sexual contentment adjustment;
general contentment adjustment;
vital sign adjustment;
cholesterol adjustment;
vitamin D adjustment.
chronic disease adjustment.

12. A method for a health wellness assessment and management, comprising:
receiving, by a processor, a current body composition analysis for a subject;
receiving, by the processor, a current lab test including a testosterone measurement for the subject;
receiving, by the processor, a current questionnaire response including a chronological age for the subject;
computing, by the processor, a current bio-wellness age for the subject based on the current body composition analysis, the current lab test, and the current questionnaire response and communicating the current bio-wellness age to the subject as a gamification parameter to motivate and monitor the subject's participation in health wellness improvement programs;
generating, by the processor, a customized health and wellness plan including a target testosterone level and a target bio-wellness age based on the target testosterone level for the subject and communicating the customized health and wellness plan to the subject;
generating, by the processor, a testosterone replacement therapy selected to cause the subject to attain the target testosterone level and communicating the testosterone replacement therapy to the subject.

13. The method of claim 12, further comprising:
receiving, by the processor, a subsequent body composition analysis for the subject;
receiving, by the processor, a subsequent testosterone measurement for the subject;
computing, by the processor, a subsequent bio-wellness age for the subject based on the subsequent body composition analysis and the testosterone measurement and communicating the subsequent bio-wellness age to the subject.

14. The method of claim 12, further comprising computing, by the processor, the current bio-wellness age based on the testosterone level for the subject and typical testosterone level for age-based categories of men.

15. The method of claim 12, further comprising computing, by the processor, the current bio-wellness age based on the testosterone level for the subject and typical testosterone level for age-based categories of men adjusted by one or more of the following:
body fat adjustment;
facial wrinkle adjustment;
waistline adjustment;
visceral fat adjustment;
sleep adjustment;
smoking adjustment;
alcohol adjustment;
stress adjustment;
exercise adjustment;
nutrition adjustment;
sexual contentment adjustment;
general contentment adjustment;
vital sign adjustment;
cholesterol adjustment;
vitamin D adjustment.
chronic disease adjustment.

16. A method for a health wellness assessment and management, comprising:
receiving a current body composition analysis for a subject;
receiving a current lab test including a testosterone measurement for the subject;
receiving a current questionnaire response including a chronological age for the subject;
computing a current bio-wellness age for the subject based on the current body composition analysis, the current lab test, and the current questionnaire response and communicating the current bio-wellness age to the subject as a gamification parameter to motivate and monitor the subject's participation in health wellness improvement programs;
generating a customized health and wellness plan including a target testosterone level and a target bio-wellness age based on the target testosterone level for the subject and communicating the customized health and wellness plan to the subject;
generating a testosterone replacement therapy selected to cause the subject to attain the target testosterone level and communicating the testosterone replacement therapy to the subject;
receiving a subsequent body composition analysis for the subject;
receiving a subsequent testosterone measurement for the subject;
computing a subsequent bio-wellness age for the subject based on the subsequent body composition analysis and the testosterone measurement and communicating the subsequent bio-wellness age to the subject as a subsequent gamification parameter to motivate and monitor the subject's participation in health wellness improvement programs.

17. The computer storage medium of claim 16, further comprising:
receiving another current questionnaire response including current exercise information for the subject and computing the current bio-wellness age based on the current exercise information for the subject;
generating target exercise information for the subject and computing the target bio-wellness age based on the target exercise information for the subject.

18. The computer storage medium of claim 16, further comprising:
receiving another current questionnaire response including current nutrition information for the subject and computing the current bio-wellness age based on the current nutrition information for the subject;
generating target nutrition information for the subject and computing the target bio-wellness age based on the target nutrition information for the subject.

19. The computer storage medium of claim 16, further comprising:
receiving another current questionnaire response including current sexual contentment information for the subject and computing the current bio-wellness age based on the current sexual contentment information for the subject;
generating target sexual contentment information for the subject and computing the target bio-wellness age based on the target sexual contentment information for the subject.

20. The computer storage medium of claim 16, further comprising:
receiving current body fat information for the subject and computing the current bio-wellness age based on the current body fat information for the subject;
generating target body fat information for the subject and computing the target bio-wellness age based on the target body fat information for the subject.
